(19) United States
(12) Patent Application Publication
(10) Pub. No.: US 2026/0295268 A1
(43) Pub. Date: Oct. 1, 2026

(54) SYSTEMS AND METHODS FOR IMPROVING EXTERNALLY POWERED CORTICAL STIMULATION (XCS) WAVEFORMS

(71) Applicant: Motif Neurotech, Inc., Houston, TX (US)

(72) Inventors: Brandon Thio, Houston, TX (US); Benjamin Ferleger, Houston, TX (US); Ameya Nanivadekar, Houston, TX (US); Landan Mintch, Houston, TX (US); Amanda Singer, Houston, TX (US); Elizabeth Commissaris, Houston, TX (US); Jacob Robinson, Houston, TX (US); Steven Goetz, Houston, TX (US)

(73) Assignee: Motif Neurotech, Inc., Houston, TX (US)

(21) Appl. No.: 19/559,857

(22) Filed: Mar. 6, 2026

Related U.S. Application Data
(60) Provisional application No. 63/777,566, filed on Mar. 25, 2025.
(60) Provisional application No. 63/778,146, filed on Mar. 26, 2025.

(51) Int. Cl. A61N 1/36; A61N 1/05; A61N 1/372; A61N 1/378

(52) U.S. Cl. CPC A61N 1/36139; A61N 1/0531; A61N 1/0539; A61N 1/36171; A61N 1/36178; A61N 1/37235; A61N 1/3787

(57) ABSTRACT

The present disclosure relates to techniques for improving neuromodulation and therapeutic brain interventions by leveraging a multi-layered waveform generation module designed to induce long-term neuroplasticity that is efficient and effective. The disclosed techniques may involve a waveform generation module configured to generate a stimulation waveform comprising five distinct layers, each targeting specific neural mechanisms and enhancing stimulation delivery. These layers may include neural activation, impulse response, theta modulation, and stimulation parameters such as burst cycle control, hourly stimulation optimization, and daily stimulation scheduling. The generated stimulation waveform may be customized based on neural data (e.g., EEG, ECoG, fNIRS, epidural electrocorticography or stereo-EEG data) to align with a subject's intrinsic brain activity. Furthermore, a stimulation applicator may deliver the stimulation waveform through advanced electrodes' configurations, maximizing spatial and temporal selectivity while minimizing the electrodes' wear. The disclosed techniques may further facilitate a synchronization of neural activity, supporting a synaptic recovery, and yielding lasting therapeutic benefits.

### CROSS-REFERENCE TO RELATED APPLICATIONS

This application claims the benefit of U.S. Provisional Patent Application No. 63/777,566 filed on Mar. 25, 2025, and to U.S. Provisional Patent Application No. 63/778,146 filed on Mar. 26, 2025. The entire disclosures of the aforementioned applications are incorporated by reference herein in their entireties for all purposes.

### BACKGROUND

Neuromodulation is a revolutionary therapeutic technique that employs targeted electrical stimulation to regulate activities of specific regions within the nervous system. This approach leverages the brain's inherent plasticity and adaptability, offering a non-pharmacological alternative or a complement to augment conventional treatments. Neuromodulation has demonstrated significant potential in managing a broad spectrum of neurological and psychiatric conditions including chronic pain, epilepsy, depression, Parkinson's disease, and cognitive impairments. By directly influencing neural circuits, it enables precise modulation of an aberrant brain activity, paving the way for personalized interventions.

Among the various neuromodulation techniques, externally powered cortical stimulation (XCS) has particularly emerged as a promising modality. XCS combines advanced engineering technologies with neuroscience methods to deliver electrical stimulation directly to targeted brain regions without requiring a permanent power source within a subject's body. XCS may use millimeter-sized neuromodulation devices that may be implanted epidurally in the brain through minimally invasive procedures, such as a burr-hole technique. These devices may be externally powered and controlled using a magnetic coil, enabling clinicians to program and deliver tailored stimulation patterns to specific regions of the brain.

The efficacy of neuromodulation therapies, including XCS, may be dependent on the characteristics of the electrical stimulation pattern, commonly referred to as the stimulation waveform. A well-designed stimulation waveform may influence neural activity to achieve therapeutic goals, such as enhancing long-term neuroplasticity, mitigating symptoms of neurological diseases, or promoting functional recovery. But achieving these therapeutic outcomes effectively still remains challenging due to the complex and dynamic nature of the brain's neural networks.

Traditional neuromodulation therapies continuously deliver stimulation to engage or block neural activity but often may not be able to provide lasting therapeutic benefits once turned off. Moreover, these therapies often rely on generalized patterns, overlooking variability in spatial and temporal dynamics of targeted neurons. As a result, the effectiveness of these therapies may vary significantly across individuals, and their potential for achieving long-lasting neuroplastic changes may remain underutilized. Therefore, innovative techniques to enhance stimulation waveforms for engaging specific neurophysiological mechanisms and target distinct neurons may be effective.

### SUMMARY

Some embodiments of the present disclosure relate to techniques for generating a stimulation waveform by incorporating neural activity patterns of targeted neurons and implementing structured stimulation schedules to achieve long-term neuroplasticity. The generation of the stimulation waveform may include multiple levels, each introducing distinct frequency components tailored to target specific neural mechanisms and enhance neuroplasticity.

According to some aspects, neural data of a subject may be obtained (in real-time) to analyze neural activity, identify neural activity patterns, and determine appropriate schedule parameters for generating a tailored stimulation waveform. The neural data may be obtained using a sensing apparatus comprising one or more sensors, a communication module, and a stimulation applicator. The one or more sensors imbedded into the sensing apparatus may include electroencephalogram (EEG) electrodes, epidural electrocorticography electrodes, electrocorticography (ECoG) electrodes, functional near infrared spectroscopy (fNIRS), ultrasound (US), and/or stereo-electroencephalogram (stereo-EEG) electrodes. In some instances, the sensing apparatus may be a headgear, a headset, a head-patch housing the stimulation applicator, the one or more sensors and the communication module collectively to enable wireless communication with a computing device. The communication module may include a wireless transceiver configured to receive control signals from the computing system for adjusting the stimulation waveform and an external transmitter coil for transferring power to the receiver coil of an implantable device. Moreover, the stimulation applicator may be configured to communicate with the implantable device wirelessly for controlling and administering the stimulations. The implantable device includes a receiver coil, a power management module, and a stimulation controller for administering the stimulation to the targeted neurons. The receiver coil wirelessly receives an alternating magnetic field from the external transmitter coil within the sensing apparatus, generating an alternating current (AC) via inductive coupling. The power management module then rectifies and regulates this AC into direct current (DC) to ensure a stable power supply for the implantable device. The stimulation controller, connected to the power module and stimulation applicator, dynamically adjusts the stimulation waveform based on real-time neural feedback.

Typically, millimeter-sized stimulation electrodes may be implanted inside the skull through a burr-hole procedure and powered externally using a magnetic coil that may also enable digital programming. The configuration of the stimulation electrodes may vary based on the stimulation requirements. For instance, a single dural-facing contact or multiple contacts, which can be shorted together, may be employed. For targeted stimulation of small regions (e.g., stimulating the motor cortex to improve motor recovery in stroke patients, deep brain stimulation (DBS) for focused effects in Parkinson's disease, or targeting the hippocampal CA1 region to address memory deficits in Alzheimer's disease), a single stimulation electrode may be sufficient, minimizing electrode corrosion by keeping both the stimulation amplitude and pulse width to a minimal level.

In contrast, when stimulating larger areas of neural tissue (e.g., engaging widespread cortical networks to alleviate depression through transcranial direct current stimulation (tDCS), stimulating the somatosensory cortex to enhance sensory feedback in prosthetic limb users, or engaging the prefrontal cortex to treat conditions like Attention Deficit Hyperactivity Disorder (ADHD) or Post-Traumatic Stress Disorder (PTSD), multiple stimulation electrodes may be shorted together. This configuration allows the overall stimulation amplitude and pulse width to be increased as needed while distributing the stimulation delivery across electrodes. By operating below the corrosion threshold for each electrode, the distributed stimulation may activate a broader tissue area within a shorter period of time.

In some aspects, the delivery of stimulation may alternate between a single stimulation electrode and multiple electrodes, which can be shorted together, enabling dynamic modulation of neural activity for comprehensive therapeutic effects. For example, in epilepsy treatment, a single electrode may initially deliver targeted stimulation to the focal seizure onset zone, suppressing localized activity. Subsequently, switching to multiple shorted electrodes may engage a broader network of surrounding neurons, stabilizing the overall neural activity, and preventing the spread of epileptiform discharges. Similarly, in stroke rehabilitation, the dynamic alternation between configurations may allow specific motor regions to be stimulated with a single electrode to enhance localized motor control. The stimulation may then shift to multiple shorted electrodes to synchronize larger cortical areas, promoting widespread neuroplasticity and functional recovery.

The neural data of the subject may be analyzed to determine neural activity patterns of targeted neurons that may serve as the baseline frequency components for generating a stimulation waveform. The neural activity patterns of the targeted neurons may include natural frequencies, activation thresholds, theta rhythm oscillations, wavelet or spectral features of the neural data, impulse response, and target engagement metrics. By analyzing baseline neural activity, the minimum level of stimulation required to activate the targeted neurons may be determined, establishing an activation threshold. To further refine stimulation parameters, the system delivers short bursts of stimulation to the neurons and measures their impulse response, allowing for real-time adjustments in response to factors such as neuroplasticity, changes in posture, or neurodegeneration. Additionally, the system identifies a theta rhythm (neural oscillation frequency for engaging neuroplasticity) by analyzing neural data within the 3 to 8 Hz frequency range. This may be done by selecting a frequency within one standard deviation of a peak power in a power spectral density computed based on the frequency components obtained from the neural data, to obtain the theta rhythm.

The natural frequencies or maximum power band frequencies may refer to the specific frequencies at which a neuron or a group of neurons naturally oscillates when stimulated. These frequencies are intrinsic properties of neural circuits and may be identified by sweeping through stimulation frequencies while recording neural activity through the one or more sensors. When neurons are stimulated at or near their natural frequency, they may exhibit sustained oscillatory activity even after the external cortical stimulation ceases. The power and duration of neural oscillations in the frequency band corresponding to the applied stimulation frequency may serve as indicators of the natural frequency.

Thus, the principle of natural frequency-based neural activation may allow for tailored stimulation strategies, such as super-threshold stimulation at a neuron's natural frequency may enhance direct activation, and sub-threshold stimulation may facilitate entrainment, thereby modulating neuronal excitability and network dynamics in a controlled manner. For example, interneurons, which have resonant frequencies typically between 10-50 Hz, may be preferentially activated by applying super-threshold pulses at a frequency such as 25 Hz, which aligns with their natural oscillatory behavior. Similarly, pyramidal neurons, which have resonant frequencies between 2-10 Hz, may be selectively engaged using stimulation frequencies within their range. By systematically sweeping through stimulation frequencies and observing sustained oscillations in neural recordings, the natural (maximum power band) frequencies of targeted neurons may be detected, allowing for precise neuromodulation strategies in therapeutic and research applications.

Subsequently, the activation threshold may be vital frequency components for the stimulation waveform as they define the conditions under which specific neurons may be activated and engaged. The activation threshold represents the minimum stimulus required to elicit neural firing, which helps guide waveform's intensity for effective stimulation. Meanwhile, theta rhythms, typically in the 3.5-7.5 Hz range, are natural oscillations that can play an important role in facilitating neuroplasticity. The activity threshold may be determined by analyzing baseline neural activity patterns from the subject's neural data using machine-learning (ML) models.

Furthermore, in addition to the neural activity patterns, stimulation parameters for generating the stimulation waveform may also include predefined or computed schedule cycles. The schedule cycles may include burst cycles, duty cycles, hourly or daily stimulation durations.

A plurality of stimulation pulses may be generated (also referred herein as a train) based on the determined neural activity patterns (i.e., the resonant frequencies, the activity threshold, and the theta rhythm) that may further be aligned with an initial waveform. For instance, a train may comprise multiple stimulation pulses (e.g., 10 pulses at 250 Hz). The initial waveform may then be modulated to include the schedule parameters including ultra-low duty cycles with stimulation periods repeated over extended periods of time (e.g., minutes or hours). According to some aspects of the present disclosure, resulting waveform may be further modulated to include a burst that comprises multiple trains (e.g., 10 trains at 5 Hz). Following this, a duty cycle may may be introduced into the resulting waveform that includes include multiple bursts (e.g., 60 bursts at 0.1 Hz). Subsequently, an hourly cycle comprising of multiple duty cycles (e.g., 10 duty cycles delivered once per hour for 10 hours) may be includes in the resulting waveform through further modulation. Short bursts of stimulation (e.g., 5-15 minutes every hour for up to 10 hours per day) may provide off-time periods that may be necessary for forming and maturing of dendritic spines. These dendritic spines may be significant for neuroplastic changes, as their formation encodes durable plasticity in the brain.

Finally, incorporating daily stimulation durations of just a few minutes per day may be essential for firmly establishing neuroplasticity over several days. A daily cycle may include multiple hourly cycles (e.g., 5 hourly cycles delivered once per day for 5 days). According to some aspects, additional layers of waveform generation, involving stimulation cycles with even longer frequencies spanning over weeks or months, may also be introduced to enhance long-term neuroplastic changes. Once the stimulation waveform is modulated to integrate hourly and daily stimulation cycles, it may be delivered to the targeted neurons via the implantable device comprising the one or more stimulation electrodes positioned on the dural surface of the subject's skull.

Changes in neural response to stimulation may be evaluated using machine-learning (ML) models to gain insights into neural response patterns and subsequently optimize a planned therapy. Engagement metrics, including firing rates, synaptic strength, stimulation engagement, and evoked potentials of the targeted neurons, may be measured in real-time through the one or more sensors. To determine the firing rate, the system detects action potentials (spikes) within a specific time window using spike-sorting algorithms, spectral analysis, or wavelet transforms to isolate neural firing events. Similarly, synaptic strength may be estimated by examining variations in the amplitude and duration of neural signals over time.

Additionally, by measuring spectral power changes in the on gamma (30-100 Hz) and high-gamma (>100 Hz) frequency bands, the system assesses neural responses to stimulation and adjusts parameters accordingly. These adjustments may help enhance neural synchronization, improve sensory perception, strengthen working memory, optimize cortical processing, and regulate synaptic activity, ensuring effective and adaptive neuromodulation.

Furthermore, neural activity is highly dynamic, influenced by internal and external factors such as synaptic plasticity, fatigue, behavioral state shifts, and even positional adjustments of the head or body. For example, stimulation engagement assesses whether neurons are actively responding to the applied stimulation. If stimulation engagement weakens over time due to adaptation, synaptic fatigue, or neurodegeneration, the system can modify parameters such as intensity, pulse width, or stimulation frequency to restore neural excitability. Additionally, by comparing real-time target engagement metrics to predefined thresholds, the system can determine whether stimulation is within the expected physiological range or whether adjustments are needed to maintain efficacy. These real-time measurements may provide vital feedback for dynamically adjusting the stimulation parameters to optimize therapeutic outcomes. For instance, if the measured activity metrics indicate insufficient neuroplasticity (e.g., firing rates or synaptic strength below expected thresholds), the stimulation parameters may be increased to enhance neuroplasticity. Conversely, if the measured metrics exceed optimal thresholds, the stimulation parameters may be reduced to avoid overstimulation and promote stability. Once neuroplasticity may have been established, the frequency and duration of stimulation cycles may be reduced, allowing for the maintenance of neuroplasticity with less frequent interventions.

In some embodiments, a system is provided that includes one or more data processors and a non-transitory computer readable storage medium containing instruction which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1A illustrates an overview of a neuromodulation system to engage neuroplasticity by utilizing neural data of a subject, in accordance with various aspects of the present disclosure.

FIG. 1B illustrates an exemplary block diagram of an engagement testing system for real-time adjustment of stimulation parameters in accordance with some embodiments of the present disclosure.

FIG. 2A illustrates an example placement of a sensing apparatus comprising of one or more sensors on a subject's head to capture neural data in accordance with some embodiments of the present disclosure.

FIG. 2B illustrates an example placement of one or more stimulation electrodes configured to deliver electrical stimulation to a subject in accordance with some embodiments of the present disclosure.

FIG. 2C illustrates an example configuration of the one or more stimulation electrodes shorted together in accordance with some embodiments of the present disclosure.

FIG. 3 illustrates an exemplary block diagram of a waveform generation module configured to generate a composite waveform to engage neuroplasticity.

FIG. 4 shows an example block diagram of a stimulation applicator for delivering an electrical stimulation to a targeted region of the brain in accordance with some embodiments of the present disclosure.

FIG. 5 illustrates an example implementation of a super-threshold stimulator configured to perform super-threshold stimulation enabling targeted neural activation in accordance with some embodiments of the present disclosure.

FIG. 6 shows an example implementation of the waveform generation module and the stimulation applicator configured to perform sub-threshold stimulation to entrain a network of neurons in accordance with some embodiments of the present disclosure.

FIG. 7 shows example plots of an initial waveform and the stimulation pulses based on the baseline activity thresholds of the target neurons according to some embodiments of the present disclosure.

FIG. 8 shows example plots of the initial waveform modulated over-time based on varying modulation schemes according to some embodiments of the present disclosure.

FIG. 9 shows an example flowchart for generating a stimulation waveform based on the neural data in accordance with some embodiments of the present disclosure.

### DETAILED DESCRIPTION

The present disclosure relates to techniques for enhancing neuromodulation and therapeutic neural interventions through a multi-layered waveform generation system designed to induce long-term neuroplasticity. Each layer of the system may refine a stimulation waveform to address specific neural dynamics, such as aligning with natural activity patterns of neurons or enhancing stimulation patterns for efficiency and recovery. Together, the layers may be integrated to produce the stimulation waveform that may synchronize with the neural activity, promote targeted neuroplastic changes, and enhance therapeutic outcomes in a defined and controlled manner.

According to some aspects, neural data of a subject may be obtained (in real-time or post hoc) to analyze neural activity, identify neural activity patterns, and determine appropriate schedule parameters for generating a tailored stimulation waveform. The neural data may be obtained using a sensing apparatus comprising one or more sensors, a communication module, and a stimulation applicator. The one or more sensors imbedded into the sensing apparatus may include electroencephalogram (EEG) electrodes, epidural electrocorticography electrodes, electrocorticography (ECoG) electrodes, functional near infrared spectroscopy (fNIRS), ultrasound (US), and/or stereo-electroencephalogram (stereo-EEG) electrodes. In some instances, the sensing apparatus may be a headgear, a headset, a head-patch housing the stimulation applicator, the one or more sensors and the communication module collectively to enable wireless communication with a computing device. The communication module may include a wireless transceiver configured to receive control signals from the computing system for adjusting the stimulation waveform, whereas the stimulation applicator may be configured to communicate with the implantable device wirelessly for controlling and administering the stimulations.

The computing device may be a device (e.g., smartwatch, cell phone, tablet, laptop, etc.) operated by a clinician or a subject or a cloud computing system. The computing device may offer two distinct pathways for defining stimulation parameters: automatic determination and user-defined input, both of which culminate in the production of a composite stimulation waveform through a multi-layered workflow process.

According to some aspects of the disclosure, the computing system may autonomously analyze neural data such as EEG, ECoG, or fNIRS, ultrasound (US), stereo-EEG, or epidural electrocorticography electrodes data to extract neural activity patterns, including impulse response, activation thresholds, and theta rhythm of targeted neurons, as well as target engagement metrics and wavelet or spectral features of the neural data. For example, the system may compute activation thresholds by analyzing the power spectral density of the neural data and determine the minimum amplitude required to engage the targeted neurons. Similarly, the system may identify theta rhythm by detecting natural oscillatory patterns within the 4-8 Hz frequency range and selecting the frequency with the highest power to establish the baseline rhythm. For instance, if the analysis reveals a theta rhythm of 6 Hz and an activation threshold of 50 μA, these values may be incorporated as baseline parameters for a stimulation waveform.

Moreover, the system may determine the impulse response of the targeted neurons by probing a group of neurons through controlled stimulations, sweeping through a range of stimulation intensities and frequencies (e.g., 1-100 Hz), and recording neural activity to identify peak oscillatory responses. The impulse response, representing the minimal stimulation level required to activate the targeted neurons, may be measured by analyzing baseline neural activity patterns, wavelet or spectral features of the neural data, and transient neural responses before sustained stimulation is applied. Neurons with natural or maximum power band frequencies near the applied stimulation frequency may continue to oscillate at that frequency even after stimulation ceases. The system may analyze the power and duration of neural oscillations at different frequencies to identify the dominant natural frequencies of specific neuron populations. For example, if interneurons exhibit peak oscillatory activity at 25 Hz and pyramidal neurons at 6 Hz, these values may be incorporated into the stimulation parameters to enhance selective engagement of the targeted neurons.

Before initiating stimulation, the system may test target engagement metrics as a feedback signal to alter or improve the effectiveness of stimulation parameters. This may include assessing firing rates, synaptic strength, the effectiveness of stimulation in eliciting a measurable neural response (stimulation engagement), and oscillatory changes in spectral power within relevant frequency bands (e.g., gamma or high-gamma). Stimulation engagement refers to the extent to which the applied stimulation successfully activates or modulates the intended neural circuits, ensuring that the desired physiological response is achieved. If the target engagement metrics indicate insufficient neural activation, the system may adaptively refine the stimulation waveform to enhance its efficacy. To further assess synaptic strength and network connectivity, short bursts of stimulation may be applied while analyzing changes in evoked potentials, gamma/high-gamma spectral power, and time-series decomposition features such as short-time Fourier transform (STFT) and wavelet transforms. By incorporating pre-stimulation engagement testing, the system can adjust stimulation to engage functionally relevant neural pathways while minimizing unnecessary activation or overstimulation.

In some embodiments, the computing system may determine stimulation parameters that regulate the timing, frequency, and duration of stimulation bursts throughout waveform generation, ensuring alignment with therapeutic objectives. These parameters may include duty cycles, burst cycles lasting seconds, on/off cycles (also referred to herein as the duty cycle) spanning minutes, or multiple cycles extending over hours or days.

The term “duty cycle” refers to the proportion of time during which stimulation is actively applied versus the time when it is not. By adjusting the duty cycle, the user may control the overall intensity and frequency of stimulation. Similarly, the term “burst cycle” refers to the number of stimulation pulses delivered within a burst and the interval between bursts.

Machine-learning (ML) algorithms may be employed by the computing system to analyze neural data collected from a subject to predict the most effective stimulation parameters without intervention of a user or clinician. The computing system may leverage ML models such as supervised learning algorithms, including regression models, random forests, or neural networks. The ML models may be trained on a large dataset composed of labeled neural data. The dataset may include records of neural oscillations, activation thresholds, and the corresponding optimal stimulation parameters, gathered from prior clinical studies or real-world applications. Additionally, the ML models may learn patterns by correlating features in the neural data, such as power spectral density (PSD), peak frequencies, and burst activity, with stimulation outcomes like enhanced neuroplasticity or symptom alleviation. Advanced techniques, such as deep learning with convolutional neural networks (CNNs) for spatial patterns or recurrent neural networks (RNNs) for temporal dependencies, may be incorporated to handle complex neural dynamics.

Once trained, the ML models may process the subject's neural data in real time to compute relevant neural activity patterns and predict duty cycles, hourly and daily duration, by analyzing trends in the neural data, such as patterns of neural fatigue, cognitive engagement, or circadian rhythms. The ML model may predict a duty cycle that can balance the need for stimulation with periods of recovery to prevent overstimulation or neural adaptation. After the user's approval, the computing system may automatically forward the stimulation parameters (i.e., activation threshold, theta rhythm, duty cycles, hourly and daily durations) to the waveform generation module.

In some embodiments, the stimulation parameters may be defined by a clinician or a user through a user-interface (UI) based on the neural activity patterns observed. The UI may display the neural data in real-time through visual representations such as spectrograms, oscillatory frequency graphs, or threshold amplitude plots, enabling the clinician to make informed decisions.

Once the user has selected the appropriate stimulation parameters for the duty cycle, burst cycle, hourly and daily stimulation durations, and intensity, they may be passed to a waveform generation module. The waveform generation module may employ methods to synthesize a composite stimulation waveform that may incorporate both the stimulation parameters and the neural activity patterns, tailored to the patient's specific neural patterns and therapeutic goals.

The waveform generation module may abstract the stimulation waveform into multiple layers, each tailored to achieve a specific functional objective. For instance, the first layer may align an initial waveform with the activation threshold of targeted neurons to dynamically adapt to ongoing endogenous activity, enhancing spatial selectivity and enabling precise targeting of neurons. The second layer may modulate the waveform generated in the first layer with theta rhythms derived from the subject's neural data. By timing stimulation in relation to oscillatory cycles associated with cognitive functions such as learning and memory, the second layer enables conditions for effective therapeutic effects.

Next, a third layer may introduce rhythmic bursts with predefined duty cycles into the theta-modulated waveform that may enable subthreshold synaptic activity, promoting synergistic neural engagement and facilitating long-term potentiation. The fourth layer may align the waveform with higher-level temporal structures, such as alternating stimulation periods, for example, 15 minutes of active stimulation followed by 45 minutes of rest per hour. This may be achieved by incorporating rest intervals, which help prevent overstimulation while preserving therapeutic efficacy. Finally, the fifth layer may align hourly cycles into broader daily treatment schedules (e.g., delivering stimulation from 9 AM to 3 PM), supporting long-term therapeutic outcomes.

Following the generation of the stimulation waveform, it may be delivered to the subject via an implantable device strategically positioned on the dural surface within the skull of the subject, in a proximity to the targeted neurons. The implantable device may include one or more stimulation electrodes configured to provide the stimulation waveform to the targeted neurons. The configuration and placement of the implantable device may vary based on the subject-specific neural target and therapeutic objectives.

Furthermore, the targeted neurons or the target tissue for the stimulation may not need be limited to the brain. The stimulation waveform may be applied to other regions of the body where neural or muscular activity plays a significant part in therapeutic outcomes. For instance, peripheral nerves in the limbs may be targeted for treating conditions such as chronic pain, neuropathy, or motor impairments. Similarly, stimulation of the spinal cord may be employed for pain management or restoring motor function in cases involving spinal injury. In another example, stimulation applied to the vagus nerve (a component of the autonomic nervous system, extending from the brainstem to various organs in the chest and abdomen) may aid in managing conditions like epilepsy, depression, or inflammatory diseases by modulating autonomic nervous system activity. The positioning and configuration of the stimulation electrodes in these cases may be adapted to enhance stimulation delivery to the specific target tissues, enabling precise and effective therapy tailored to the subject's health conditions and needs.

Furthermore, changes induced by the applied stimulation may continuously be accessed by the system to enhance therapeutic outcomes. This may include accessing the engagement metrics that serve as an important feedback to help determine how effectively neural stimulation is interacting with the targeted neurons. These metrics quantify real-time neural responses, ensuring that stimulation achieves the desired physiological and therapeutic effects. Among the most important engagement metrics are impulse response, firing rates, stimulation engagement, evoked potentials, and synaptic strength, each providing unique insights into neural circuit activity. By continuously monitoring these parameters, the system can dynamically adapt stimulation settings to account for changes in neural response due to neuroplasticity, synaptic adaptation, posture shifts, or other physiological variations.

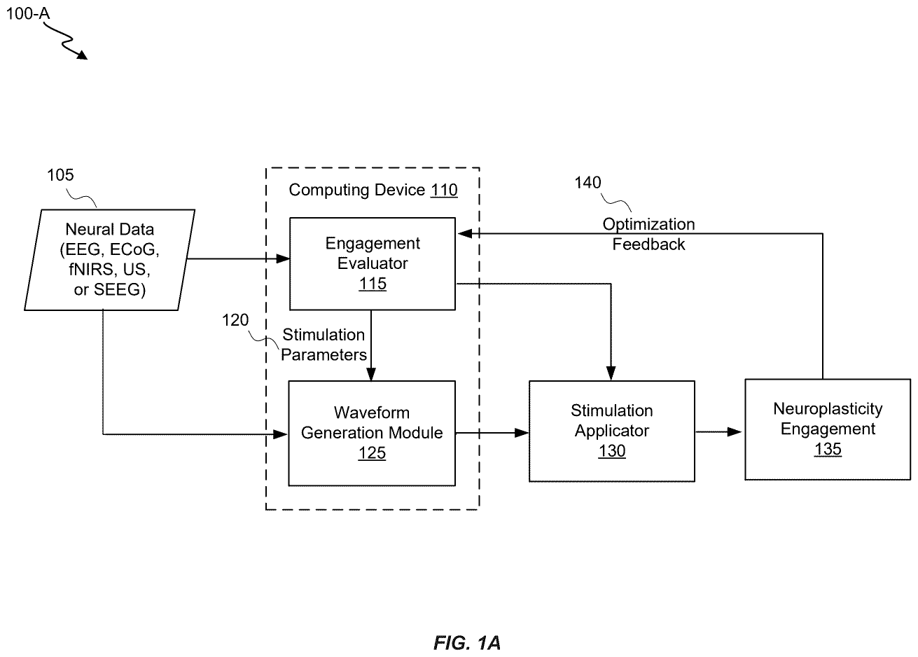

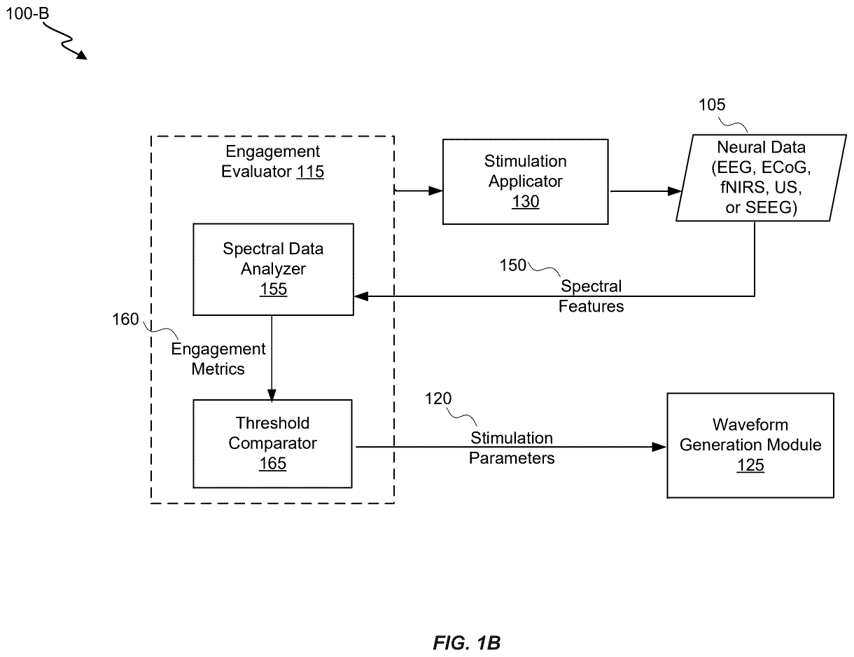

FIG. 1 illustrates an overview 100 of a neuromodulation system to engage neuroplasticity by utilizing the neural data of a subject, in accordance with various aspects of the present disclosure. The overview 100 may include neural data 105, a computing device 110, an engagement evaluator 115, a waveform generation module 125, a stimulation applicator 130, and neuroplasticity engagement 135. The neural data 105 obtained from the subject may include various forms of electrophysiological or neural signals that may provide insights into the subject's neural activity. The neural signals may include electroencephalography (EEG) data that records electrical activity from the scalp; electrocorticography (ECoG) data that captures cortical surface activity; stereo-electroencephalography (sEEG) data that measures deep brain activity, functional near-infrared spectroscopy (fNIRS) data that detects hemodynamic responses associated with neural activity; epidural electrocorticography data, or ultrasound-based neural imaging data that assesses brain activity through transcranial or implanted ultrasound sensors.

The neural data 105 obtained from the subject may be transmitted to the computing device 110 for analysis. The transmission may occur via a secure wired or wireless connection, depending on the specific design of the sensing apparatus and the computing device 110. Once the neural data is received at the computing device 110, the neural data 105 may undergo preprocessing steps, such as artifact removal, baseline correction, and signal filtering to achieve better accuracy. Advanced signal processing techniques, including Fourier transforms or wavelet decompositions, may be applied to decompose the neural data 105 into its constituent frequency bands. The preprocessing steps may enable the conversion of raw data into a form that may be suitable for machine-learning (ML) analysis.

According to some aspects, the computing device 110 may utilize the engagement evaluator 115 to determine appropriate stimulation parameters 120 based on targeted engagement testing by applying short bursts of stimulation using the stimulation applicator 130. By taking real-time feedback from the neural data 105, various spectral features may be analyzed in response to the testing stimulations to determine factors such as synaptic plasticity, impulse response, and/or activation thresholds. Additionally, other factors such as adaptation, neurodegeneration, and external influences like postural shifts may also alter how neurons respond to stimulation over time. If engagement metrics indicate a decline in neural responsiveness, the system can dynamically adjust stimulation parameters to restore optimal engagement. Thus, such a closed-loop adaptation allows for personalized and self-optimizing neuromodulation, improving the efficacy and long-term stability of the therapy. Based on the determined neural activity patterns, appropriate stimulation parameters 120 may be selected and forwarded to the waveform generation module 125. Based on the selected stimulation parameters 120, the waveform generation module 125 may construct a customized stimulation waveform, for therapeutic objectives. In some instances, the neural data 105 may be presented to a user or a clinician through the user-interface, enabling real-time visualization and analysis of the subject's neural activity. The interface may display processed neural data 105 in various formats, such as power spectral density plots, oscillatory pattern graphs, or temporal activity charts to provide intuitive insights into the subject's neural state. Based on this analysis, the user may select the stimulation parameters 120 including duty cycles, burst cycles, hourly or daily stimulation durations, neural activity patterns, or other user-defined inputs.

The waveform generation module 125 may utilize the stimulation parameters 120 provided by the user or automatically derived from the neural data 105 to construct a composite stimulation waveform. This process may involve multiple layers, where each layer may contribute to refining the stimulation waveform to achieve specific therapeutic objectives. For instance, the initial layer may address neural activation thresholds, aligning the stimulation to the endogenous activity of targeted neurons. Subsequent layers may integrate other neural activity patterns such as theta rhythms, rhythmic burst cycles with defined duty cycles, and synchronize the stimulation with hourly or daily durations as specified in the stimulation parameters 120.

The resulting composite waveform may then be transmitted to a stimulation applicator 130, configured to deliver the stimulation waveform to the target neural regions with a desired precision. The stimulation may facilitate the neuroplasticity engagement 135 by modulating neural circuits, enhancing connectivity of the neural network of the brain or promoting functional recovery based on the therapeutic goals. Additionally, the neuroplasticity engagement 135 may also enable forming new synaptic pathways, strengthening, or weakening of existing connections, and even the generation of new neurons in certain regions that support neurogenesis. For example, when a subject learns a new skill, such as playing a musical instrument, repeated practice stimulates the growth and reinforcement of neural circuits associated with motor control, memory, and auditory processing. Similarly, in rehabilitation following a stroke, targeted physical and cognitive therapy may engage neuroplasticity to help healthy brain tissue compensate for functions lost because of damaged neurons elsewhere, enabling the recovery of speech or motor abilities. Neuroplasticity engagement 135 may also play a significant role in habit formation, adaptation to sensory loss (e.g., heightened tactile sensitivity in blind individuals), and recovery from traumatic brain injuries.

Moreover, an optimization feedback 140 from the neuroplasticity engagement 135 may be transmitted back to the engagement evaluator 115, allowing for dynamic adjustments to the stimulation process. The optimization feedback 140 may include real-time changes in neural data, responses to stimulation, or target engagement metrics. These metrics quantify real-time neural responses, ensuring that stimulation achieves the desired physiological and therapeutic effects. Some of the key engagement metrics include impulse response, firing rates, stimulation engagement, evoked potentials, and synaptic strength, each offering distinct insights into neural circuit activity. By continuously monitoring these parameters, the engagement evaluator 115 can dynamically adapt stimulation settings to account for changes in neural response due to neuroplasticity, synaptic adaptation, posture shifts, or other physiological variations.

FIG. 1B illustrates an exemplary block diagram of an engagement evaluator for real-time adjustment of stimulation parameters in accordance with some embodiments of the present disclosure. The engagement evaluator 145 may include a spectral data analyzer 155 and a threshold comparator 165. The engagement evaluator 145 may send instructions to the stimulation applicator 130 to perform test stimulations on targeted neurons. The spectral features 150 obtained as a result of the applied stimulation may be sent back to the spectral data analyzer 155 for further processing. By measuring engagement metrics 160 such as gamma and high-gamma frequency bands, firing rates, stimulation engagement, evoked potentials, and synaptic strength, the engagement evaluator 145 may assess how neurons respond to stimulation. The engagement metrics 160 may then be forwarded to the threshold comparator 165, which evaluates changes in neural dynamics and suggests optimized stimulation parameters 120 for the waveform generation module 125.

Various machine learning (ML) models may be employed by the threshold comparator 165 for analyzing and comparing the engagement metrics 160 to refine stimulation parameters 160. By comparing real-time engagement metrics to predefined thresholds, the threshold comparator 165 can detect changes in neural activity using equations such as:

$$Δ M = {M}_{measured} - {M}_{threshold} , \qquad ( Eq . 1 )$$

where M represents firing rate (FR), synaptic strength (SS), or impulse response (IR). Neuroplasticity is detected when:

$$\vert Δ M \vert > ϵ$$

where ∈ is a predefined sensitivity parameter.

Neural activity is highly dynamic and influenced by synaptic plasticity, fatigue, behavioral shifts, changes in posture, and other physiological or environmental factors. Stimulation engagement is a critical metric for assessing whether neurons actively respond to the applied stimulation and if they do so in a predictable fashion. If engagement weakens over time due to factors like adaptation, synaptic fatigue, or neurodegeneration, the system can dynamically adjust stimulation parameters 120, such as intensity, pulse width, or frequency, to restore neural excitability. Additionally, real-time comparison of target engagement metrics with predefined thresholds helps to maintain stimulation within the expected physiological range. If the impulse response indicates a shift in neuronal excitability, it may signal underlying neuroplastic changes or non-plasticity-driven variations, such as positional adjustments affecting cerebrospinal fluid dynamics or skull-brain interface properties. This adaptability facilitates the maintenance consistent therapeutic outcomes even in the presence of external influences.

Closed-loop stimulation relies on engagement metrics 160 to enhance neuromodulation and optimize therapy delivery. If firing rates decrease over time due to homeostatic downregulation or synaptic fatigue, the engagement evaluator 115 may compensate by adjusting burst frequency or intensity. Similarly, monitoring oscillatory power in specific frequency bands such as theta (3-8 Hz) and gamma (>30 Hz) may help in effectively entraining neural circuits. If initial stimulation enhances gamma power, but later declines due to adaptation, the engagement evaluator 115 can dynamically fine-tune stimulation parameters to restore the desired oscillatory state. Furthermore, synaptic strength, measured through evoked potentials, provides insights into long-term potentiation (LTP) and long-term depression (LTD), allowing the system to reinforce or counteract these plasticity-driven changes as needed.

Unsupervised models, such as k-means clustering and PCA, identify patterns in neural responses without predefined labels, revealing groups of neurons with similar response characteristics. Additionally, reinforcement learning models, including Q-learning and deep reinforcement learning, iteratively refine the stimulation parameters 160 by treating neural response as feedback. These models maximize reward functions based on sustained neuroplasticity, improved cognitive function, or reduced pathological activity. For example, a reinforcement learning algorithm may adjust frequency and amplitude in real-time to optimize theta rhythm synchrony. The adjusted stimulation parameters 160 may be sent to the waveform generation module 125 for further processing.

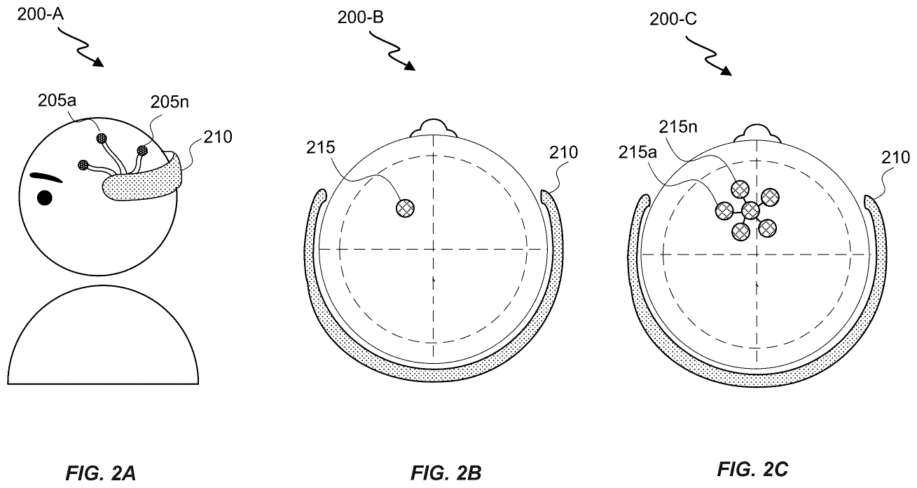

FIG. 2A illustrates an example placement of a sensing apparatus comprising of one or more sensors on a subject's head to capture the neural data 105 in accordance with some embodiments of the present disclosure. The sensing apparatus may include one or more sensors 205a-n adhered to a headgear 210 to capture the neural data 105. The one or more sensors 205a-n may include active electrodes and reference electrodes. Both active and reference electrodes may be placed close to each other but may not be electrically connected. In some instances, a bias electrode may be attached to the ear (e.g., ear lobe or back side of ear) of the subject using an electrode lead. The headgear 210 may include straps, and fasteners (e.g., Velcro, hook, button, etc.) for custom fit, adjustment, and comfort of the subject and may further include plurality of slots for attaching electrodes or electrode snap connectors at specific positions. In some embodiments, the sensing apparatus may include a cap, a head harness, a head patch, or other wearable structures that may be designed to house and secure one or more sensors 205a-n in a proximity to the subject's scalp or other neural regions of interest.

Additionally, the sensing apparatus may include integrated wiring or wireless modules to facilitate the transmission of captured neural data 105 to the computing device 110 for real-time analysis. The one or more sensors 205a-n within the sensing apparatus may be configured to monitor neural activities non-invasively, such as through electroencephalography (EEG), or invasively, such as through electrocorticography (ECoG) or stereo-EEG, depending on the intended application and therapeutic goals.

FIG. 2B illustrates an example placement of one stimulation electrode of the one or more stimulation electrodes configured to deliver electrical stimulation to the subject in accordance with some aspects of the present disclosure. The placement of the stimulation electrode 215 may vary depending on the specific therapeutic goals and the targeted neural region. In some embodiments, stimulation electrode 215 may be adhered to the surface of the subject's scalp or skin, allowing for non-invasive stimulation of underlying neural structures. This approach may be used for conditions where a broad neural engagement may be preferred.

In some embodiments, the stimulation electrode 215 may be placed inside or near the targeted neural area using minimally invasive procedures, such as a burr hole procedure. In such embodiments, the stimulation electrode may be placed epidurally. The burr hole technique may involve creating a small opening in the skull to insert the stimulation electrode 215 in a proximity to a specific neural target, such as deep brain structures or cortical regions. The burr hole procedure may also provide a precise electrode placement, enabling the delivery of electrical stimulation to highly specific targeted neurons.

When stimulating through a single electrode contact, the stimulation amplitude and pulse width may be carefully controlled to minimize electrode corrosion and extend the operational lifespan of the stimulation electrode 215. In such cases, the computing device 110 may dynamically adjust the stimulation parameters 120 to enable an effective neuroplastic engagement while maintaining the electrode's integrity. The careful modulation of stimulation parameters may also reduce the risk of unintended tissue damage or overstimulation, contributing to not only safety but also accuracy of the intervention. Additionally, by accommodating both non-invasive and invasive configurations, the computing device 110 may offer flexibility in addressing a wide range of therapeutic needs, from general neural modulation to highly targeted interventions.

FIG. 2C illustrates an example configuration of the one or more stimulation electrodes shorted together in accordance with some aspects of the present disclosure. According to the example placement, one or more stimulation electrodes 215a-n may be shorted together to encompass a larger area of the brain, making this configuration particularly suitable for conditions where broad neural engagement may be preferred. The shorted electrodes may involve stimulating with all dural-facing contacts shorted together, and rapidly oscillating stimulation between multiple dural-facing contacts either independently or through the dural-facing contacts shorted together.

When stimulating through a single electrode contact, the stimulation amplitude and pulse width may be limited to prevent electrode that may reduce the risk of tissue damage and electrode degradation, making it suitable for scenarios that may need a long-term use. However, the smaller surface area of a single electrode contact inherently limits the breadth of neural tissue that can be engaged during stimulation. Thus, in configurations involving all dural-facing electrode contacts shorted together, the overall surface area of the one or more stimulating electrodes 215a-n may increase and that may significantly raise the corrosion threshold. This allows for higher stimulation amplitudes and pulse widths compared to a single-contact stimulation. The increased electrode surface area distributes the electrical current more broadly, enabling an activation of larger neural regions, beneficial for conditions requiring extensive neural engagement, such as in treatments targeting widespread cortical or subcortical areas.

In some instances of the disclosure involves oscillating the stimulation among multiple electrode contacts of the one or more stimulating electrodes 215a-n, either independently or with shorted configurations. By rapidly alternating between one or more stimulating electrodes 215a-n, the computing device 110 may stimulate just below the corrosion threshold at several distributed locations. By leveraging distributed stimulation, a larger volume of neural tissue may be engaged, maximizing spatial selectivity and breadth of neural activation within a short period effectively. Oscillatory stimulation may also mitigate the risk of localized tissue heating and electrode degradation by distributing the electrical load across multiple contacts. For instance, a single-contact stimulation may be employed for fine-tuned neural targeting in deep brain stimulation, while shorted or oscillatory configurations may be utilized for broader cortical activation in treatments addressing diffused neurological conditions. The ability to dynamically switch between these modes may offer significant versatility, enhancing stimulation strategies for several diverse clinical scenarios.

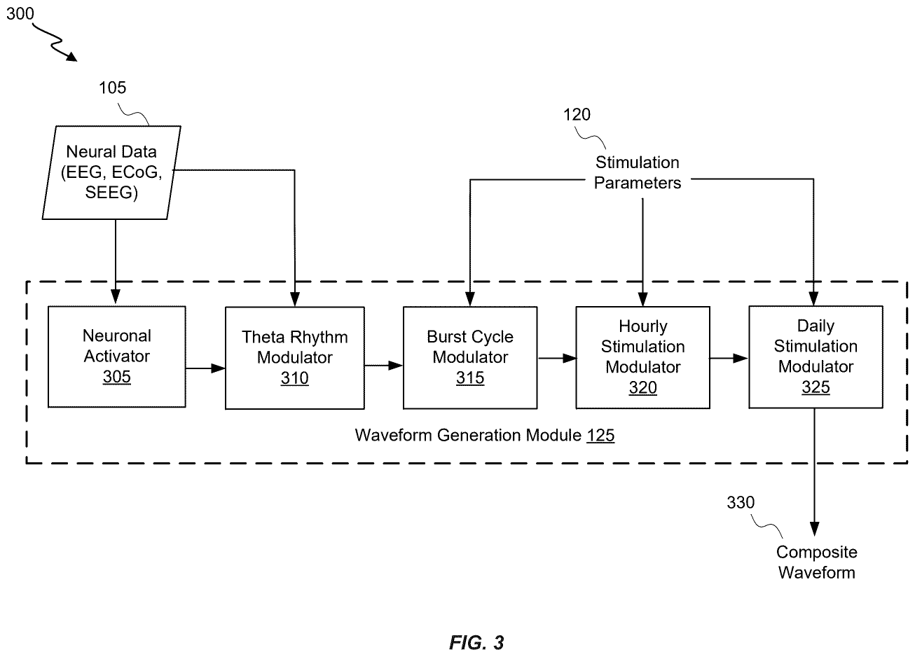

FIG. 3 illustrates an exemplary block diagram 300 of the waveform generation module 125 configured to generate a composite waveform to engage neuroplasticity. The waveform generation module 125 may include a neural activator 305, a theta rhythm modulator 310, a burst cycle modulator 315, an hourly stimulation modulator 320 and a daily stimulation modulator 325. The first layer may begin with the neural activator 305 that may take the neural data 105 of a subject as an input and generate stimulation pulses based on the baseline activity thresholds of the target neurons.

The activity thresholds may vary between individuals and may further be influenced by the ongoing endogenous neural activity. To address this variability, various ML models may be employed by the neural activator 305, such as supervised learning models like decision trees or random forests, trained on datasets containing neural activity patterns and corresponding optimal thresholds for specific conditions. For instance, in patients with an epilepsy, the ML models may analyze neural data 105 to detect pre-seizure activity and determine the appropriate thresholds for delivering stimulation to prevent a seizure. Similarly, reinforcement learning models could adaptively adjust the stimulation threshold based on real-time feedback from neural responses, making it suitable for neuroplastic modulation in conditions like depression or stroke recovery.

Once the activity threshold may be determined, the neural activator 305 may then generate high-frequency stimulation pulses designed to mimic natural neural firing patterns. According to some aspects, the stimulation pulses may comprise of 10 individual pulses, delivered at a frequency of 250 Hz, and referred to as a “train”. The train may indicate a sequence of a plurality of individual pulses delivered at a specific frequency. Furthermore, the train may serve as a means of precise neural activation, focusing on rapid, localized stimulation to initiate activity. Depending on the therapeutic requirements, the number of pulses may range from 1 to 40, and the frequencies may vary between 50 Hz and 1000 Hz, accommodating a broad spectrum of conditions.

For example, in Parkinson's disease, high-frequency stimulation pulses, targeting the basal ganglia, may help reduce motor symptoms by modulating pathological oscillations. Similarly, in cognitive disorders such as Alzheimer's disease, these pulses may be tailored to stimulate regions that may be associated with memory and learning, promoting synaptic connectivity and neuroplasticity. By delivering short, high-frequency trains of stimulation, the neural activator 305 may efficiently maximize the activation of a targeted brain region within a brief period, enhancing therapeutic outcomes across a range of neuroplasticity-driven treatments.

The waveform generated by the neural activator 305 may be sent to the theta rhythm modulator 310 for further modulation, advancing to the second layer of waveform generation. Each subsequent layer may build upon the previous, refining the waveform to align with the therapeutic goals and the subject's neural dynamics.

According to some aspects, the theta rhythm modulator 310 may represent the second layer of waveform generation within the waveform generation module 125. By leveraging machine-learning (ML) models to analyze the neural data 105, the theta rhythm modulator may determine the subject's intrinsic theta rhythm that may be a neural oscillation frequency typically within the range of 3.5-7.5 Hz. Theta rhythms are critical for engaging neuroplasticity, especially in cognitive functions such as learning and memory.

To identify subject-specific theta frequencies, ML models may analyze the neural data 105 to compute power spectral density (PSD) and extract theta frequency. Supervised learning models, such as decision trees or support vector machines, may be trained on datasets of neural activity associated with various cognitive tasks to predict an optimal theta frequency for neuroplastic engagement. Additionally, various reinforcement learning models might be employed in scenarios like personalized therapies for memory disorders, where the ML model may iteratively adjust stimulation parameters to maximize therapeutic outcomes. For example, in Alzheimer's disease or mild cognitive impairment, the identified theta frequency with the highest power may be used to enhance memory circuits; while in conditions, like depression, it may improve neural connectivity in targeted brain regions.

Once the subject's theta rhythm may be determined, the theta rhythm modulator 310 may refine the waveform generated by the neural activator 305 to align or synchronize it with the identified theta frequencies. According to some aspects, the theta modulation may introduce “bursts” that may be defined as a grouping of 10 or more trains of pulses, with each train comprising of 5-20 pulses delivered at frequencies ranging from 200 Hz to 500 Hz. The bursts may be modulated at a frequency in the range of 3-7 Hz, with a representative example being 5 Hz. This means that each burst may last for between 1-3 seconds, followed by an 8-second rest period, creating a 10-second cycle. For example, burst frequency (f_{burst}) may be defined as a function of the detected theta rhythm:

$${f}_{burst} = {max}_{f ∈ [ 4 , 8 ] Hz} P ( f ) , \qquad ( Eq . 2 )$$

where P(f) is the power spectral density (PSD) of the neural signal at frequency f. This may define how the computing device 110 selects the theta frequency with the highest power for modulation.

By introducing these rhythmic bursts, the theta rhythm modulator 310 may align the stimulation waveform with natural neural activity patterns in a neural activity, amplifying neuroplasticity-engaging effects. For instance, consistent bursts at 5 Hz may synchronize neural circuits involved in memory formation or cognitive recovery, enhancing their therapeutic efficacy. In some cases, the theta modulation may fix the bursts at a standard theta frequency, such as 5 Hz, for maintaining a consistency across subjects. Alternatively, the modulation may be tailored to the patient's intrinsic theta rhythm for precise synchronization with natural neural oscillations. The theta-modulated waveform, enriched with burst patterns refined for therapeutic goals, may then progress to the burst cycle modulator 315 for a further refinement.

The burst cycle modulator 315 may represent the third layer of waveform generation within the waveform generation module 125, introducing rhythmic bursting into the stimulation waveform. According to some aspects, the burst cycle modulator 315 may take user-defined input through the stimulation parameters 120, allowing the user or the clinician to specify burst cycles, such as the duration of stimulation and rest periods. For example, the burst cycle modulator may allow the user to define an On/Off cycle that delivers 60 bursts at 0.1 Hz (one burst every 10 seconds), with 10 minutes of stimulation every hour, leaving the remaining 50 minutes for rest. A recursive formula may be utilized define each cycle and their relation with the previous (sub-cycle):

$${T}_{cycle} = {N}_{sub} \times {T}_{sub} , \qquad ( Eq . 3 )$$

where T_{cycle} is the total duration of the higher-order cycle, N_{sub} is the number of sub-cycles, and T_{sub} is the duration of each sub-cycle. For example, if a burst cycle consists of 10 trains at 5 Hz, and a duty cycle consists of 60 bursts at 0.1 Hz, this formula formalizes how cycles may be structured.

In some embodiments, the burst cycle modulator 315 may employ machine-learning (ML) models to automatically determine the appropriate burst cycles for the stimulation waveform by analyzing the neural data 105. For instance, supervised learning models like random forests or gradient boosting machines may be trained on historical datasets containing neural activity patterns and their responses to various stimulation configurations. These ML models may also identify correlations between neural firing patterns and optimal burst cycles.

Furthermore, reinforcement learning models, such as deep Q-networks (DQNs), may also be used to iteratively learn the most effective burst cycles by maximizing reward functions that may be tied to therapeutic outcomes, such as enhanced synaptic plasticity or improved neural network synchronization. For example, in treating epilepsy, the ML models may evaluate firing patterns and detect periods of heightened excitation to determine burst cycles that prevent seizure onset. Similarly, for conditions like Parkinson's disease, the ML system may be configured to evaluate neurotransmitter replenishment rates to define burst cycles that may enhance motor control. By tailoring burst cycles to the subject's unique neural dynamics, the ML models may improve the precision and efficacy of a stimulation therapy.

The rhythmic bursting generated by the burst cycle modulator 315 may follow a pattern where stimulation occurs at a theta frequency for a short period (e.g., 1-5 seconds), followed by a longer rest period (e.g., 5-20 seconds). For instance, the On/Off cycle may consist of 60 bursts at 0.1 Hz, with each burst lasting approximately 2 seconds, followed by 8 seconds of rest. The rhythmic design may allow synapses to replenish their neurotransmitters and vesicles during the off phase of the cycle for an effective neural activation. The release of neurotransmitter may facilitate the firing of downstream neurons, which, in turn, enable long-term neuroplasticity. Thus, with sufficient neurotransmitter replenishment, synaptic activation may increase, enhancing the effectiveness of stimulation.

Furthermore, the third layer of waveform generation may facilitate two distinct types of plasticity, according to the theory of spike timing dependent plasticity (STDP). Facilitatory plasticity may occur when a neuron fires an action potential followed by the downstream neuron firing an action potential in a sequence. Depressive plasticity, on the other hand, may occur when a neuron fires an action potential, but the downstream neuron does not. The burst cycle modulator 315 may support facilitatory plasticity by creating a stimulation waveform that enables both primary and downstream neurons to fire action potentials sequentially, resulting in an effective neural modulation. The burst-modulated waveform may then be passed to the hourly stimulation modulator 320 for further modulation.

The hourly stimulation modulator 320 may represent the fourth layer of waveform generation within the waveform generation module 125. According to some aspects, the hourly stimulation modulator 320 may utilize the stimulation parameters 120 that may be defined by the user to determine the appropriate hourly duration of the stimulation waveform. Alternatively, in some instances, the hourly stimulation modulator 320 may employ machine learning (ML) models to automatically analyze the neural data 105 and determine the stimulation parameters 120 (i.e., the optimal hourly stimulation patterns) for the stimulation waveform. For instance, supervised learning models such as support vector machines (SVMs) or neural networks may be trained on the historical data, correlating hourly stimulation patterns with therapeutic outcomes like enhanced memory retention or improved motor control. Unsupervised learning models, such as clustering algorithms may group neural activity patterns to identify distinct periods when neurons are mostly receptive to stimulation. For example, during post-seizure recovery in epilepsy patients, ML models may be used to analyze EEG data to identify specific windows where stimulation enhances recovery and avoids inducing additional stress. Similarly, reinforcement learning models like proximal policy optimization (PPO) may adaptively adjust hourly stimulation durations by refining a reward function tied to neuroplasticity markers, such as changes in synaptic activity or neural synchronization. Such an approach may be beneficial in conditions like Alzheimer's disease, where the timing and duration of stimulation may need to adapt to fluctuating cognitive states, establishing a balance between therapeutic benefits and neural fatigue prevention.

Additionally, hourly modulation may involve alternating between short bursts of stimulation and longer rest periods, extending these patterns to durations lasting several minutes, for example 5-20 minutes each hour. Such a configuration may enable focused engagement of neural circuits within a specific time window, keeping the balance between activation and recovery. Over a typical day, the hourly stimulation pattern may repeat for up to 10 hours, providing sufficient intervals for meaningful neuroplastic changes.

Furthermore, the stimulation may be organized into On/Off cycles within each hourly interval. According to some aspects, a cycle may consist of 10 On/Off sessions, where each “On” period delivers bursts of stimulation, followed by a “rest” period with no stimulation. The cycles may be repeated once per hour over a total period of 10 hours, with stimulation bursts set at an exceptionally low frequency of 0.00028 Hz, meaning one burst may occur every hour. The low frequency of the stimulation may allow for the On/Off cycle to repeat once every hour, enabling the neurons to be stimulated periodically, followed by an adequate rest period, and maximizing the potential for sustained neuroplastic engagement without overloading the system.

Moreover, the longer rest periods between hourly stimulation sessions may enhance the growth and maturation of dendritic spines. Dendritic spines are small, protruding structures mostly found on the dendrites of neurons, serving as the primary sites for excitatory synaptic connections. These spines may play a pivotal role in neuroplasticity by enabling synaptic strength adjustments that may support learning, memory, and adaptation. The formation of new dendritic spines and the remodeling of existing ones may allow durable changes in the neural network, facilitating improved cognitive and motor functions.

During the rest periods, new dendritic spines may form in response to the stimulation, while existing spines may undergo morphological changes to enhance their stability and connectivity. Spine morphogenesis may enhance the encoding of long-term plastic changes in the brain. By incorporating the hourly stimulation layer, the waveform generation module 125 may enable the stimulation protocol to align with the natural processes of dendritic spine dynamics, improving the engagement of neuroplasticity. The resulting waveform may then pass to the daily stimulation modulator 325 for integration into a daily treatment schedule.

According to some aspects, the daily stimulation modulator 325 may represent the fifth layer of waveform generation within the waveform generation module 125, configured to organize the minutes of stimulation occurring every hour, as determined by the hourly stimulation modulator 320, into a comprehensive daily schedule. The daily stimulation modulator 325 may take the stimulation parameters 120 (user-defined or automatically generated) to set the daily stimulation schedule or, in some embodiments, may utilize machine-learning models to analyze the neural data 105 and suggest an optimal daily therapy protocol. The repeated stimulation sessions over consecutive days may play an important role in firmly establishing neuroplasticity. According to some aspects, the daily stimulation schedule may consist of 5 cycles of stimulation, each delivered once per day, over 5 consecutive days, at a frequency of 0.0000116 Hz (once per day). Such a low frequency value may enable sustained engagement with the targeted neural circuits across extended periods, thereby promoting durable neuroplastic adaptations. In some embodiments, frequencies as low as 0.0000116 Hz, or within a range of 0.00001 Hz to 0.00002 Hz, may be used to establish gradual neuroplastic processes over days or weeks.

Additionally, multiple days of carefully timed stimulation may be necessary for the brain to adapt and form long-lasting structural and functional changes. For instance, the daily stimulation pattern may ensure sustained engagement with the targeted neural circuits across extended periods, encouraging durable neuroplastic adaptations.

Once neuroplasticity may be firmly established, the stimulation requirements may significantly reduce. The daily stimulation modulator 325 may then adjust the daily therapy cycles, potentially decreasing the number of stimulation hours or even tapering off entirely to maintain the established plasticity, enabling adaptive therapy, balancing efficacy, and patient convenience. The output of the waveform generation module 125 may be a composite waveform 330 that me be sent to a stimulation applicator to be delivered to the subject via one or more stimulation electrodes 215a-n.

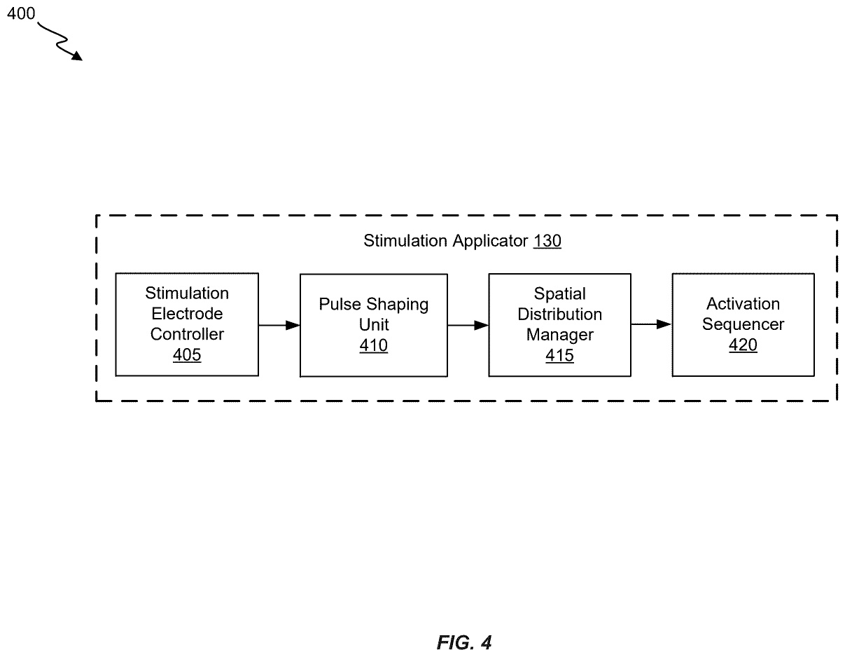

FIG. 4 shows an example block diagram 400 of a stimulation applicator 130 for delivering an electrical stimulation to a targeted region of the brain of a subject in accordance with some embodiments of the present disclosure. The stimulation applicator 130 may include an electrode controller 405, a pulse shaping unit 410, a spatial distribution manager 415, and an activation sequencer 420. The electrode controller 405 may regulate the stimulation parameters, such as amplitude, pulse width, and frequency, to ensure they accurately generate electrical signals that are suitable for the specific electrode configuration. Additionally, the electrode controller 405 may also manage the voltage and current requirements for the stimulation electrodes 215a-n to properly target the neural regions of interest.

Following the electrode controller 405, the pulse shaping unit 410 may modify the composite waveform 330 from the waveform generation module 125 by adjusting the characteristics of the stimulation pulses, such as their shape, duration, and rise/fall times, to enhance the efficacy, effectiveness, and safety of stimulation. Moreover, the pulse shaping unit 410 may fine-tune the pulse width, amplitude, and frequency to improve the stimulation's effectiveness while minimizing risks like overstimulation or damage to the tissues.

The subsequent component, spatial distribution manager 415, may determine how to distribute the stimulation across the one or more stimulation electrodes 215a-n. The spatial distribution manager 415 may enhance the spatial selectivity of the stimulation, enabling the targeted neurons to be effectively activated while minimizing unnecessary spread of the stimulation to other regions. By considering factors like electrode placement and the intended neural target area, the spatial distribution manager 415 may adjust the distribution of stimulation to maximize the effect on the targeted region or the targeted area of the brain.

Next, the activation sequencer 420 may manage the sequencing of stimulation pulses, including the precise timing and pattern of pulse delivery, to align with the therapeutic objectives. While the waveform generation module 125 defines the overall structure of the waveform, the activation sequencer 420 may operate at a more granular level, overseeing real-time pulse delivery and enabling dynamic adjustments based on ongoing feedback or variations in the subject's neural activity. Additionally, the activation sequencer 420 may support complex temporal patterns, such as alternating stimulation across the one or more stimulation electrodes 215a-n, synchronizing pulses with real-time neural oscillations, or implementing adaptive stimulation patterns, enhancing the specificity and effectiveness of the neural engagement. Moreover, the activation sequencer 420 may optimize the electrical output to align with the capabilities of the stimulation electrodes, maintaining appropriate voltage and current thresholds during the stimulation operation.

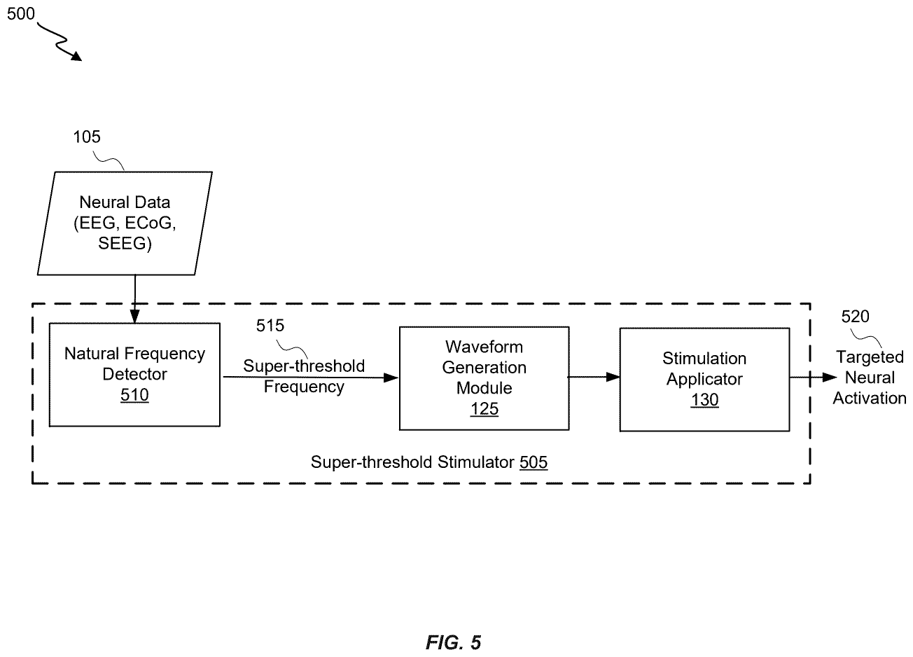

FIG. 5 illustrates an example implementation 500 of a super-threshold stimulator 505 configured to enable targeted neural activation in accordance with some embodiments of the present disclosure. The super-threshold stimulator 505 may include a resonant frequency detector 510, the waveform generation module 125, and the stimulation applicator 130. The natural frequency detector 510 may identify the unique natural frequencies or maximum power band frequencies of various types of neurons by accessing the neural data 105 of the subject to determine a super-threshold frequency 515.

Neurons are known to oscillate at intrinsic firing rates, termed natural frequencies (maximum power band frequencies), that may differ across neuronal subtypes. For example, interneurons typically resonate at higher frequencies (10-50 Hz), while pyramidal neurons may resonate at lower frequencies (2-10 Hz). To determine these frequencies, the natural frequency detector 510 may sweep through a range of stimulation frequencies (e.g., 1-100 Hz) and record the subsequent neural activity using the neural data 105 (e.g., EEG, ECoG. SEEG or epidural electrocorticography data). When the targeted neurons are stimulated at their natural frequency, the neurons may sustain oscillations for a period after the stimulation stops. By analyzing power and duration of the neural activity in the recorded data, the natural frequency detector 510 may identify peak frequencies corresponding to the natural frequencies of specific neurons and output the super-threshold frequency 515. In some aspects, machine-learning models may be used for detection of natural frequencies, for instance, convolutional neural networks (CNNs) may be employed to differentiate between subtle variations in the firing patterns of interneurons and pyramidal neurons, improving the accuracy of frequency selection.

As part of the super-threshold stimulator 505, the waveform generation module 125 may use the super-threshold frequency 515 data to generate a tailored super-threshold stimulation waveform. For instance, to preferentially activate interneurons, the waveform generation module 125 may create waveforms with pulses at 25 Hz, a frequency that interneurons may follow more effectively than pyramidal neurons. Interneurons are often inhibitory neurons that may resonate at higher frequencies, typically between 10-50 Hz, and are involved in fine-tuning and balancing excitatory inputs, whereas pyramidal neurons are a type of excitatory neurons primarily found in the cerebral cortex, hippocampus, and amygdala that typically resonate at lower frequencies, generally between 2-10 Hz, which aligns with slow oscillatory rhythms such as delta and theta waves.

The difference in natural frequencies between pyramidal neurons and interneurons may form the basis for frequency-specific neural stimulation. For example, in treating Parkinson's disease, super-threshold stimulation at specific frequencies may help reduce tremors by selectively engaging inhibitory circuits in the basal ganglia. Additionally, ML techniques such as reinforcement learning may be used to improve waveform parameters by iteratively testing and refining stimulation frequencies to achieve the best therapeutic outcome for each individual subject.

Next, the stimulation applicator 130 may be utilized to deliver the generated stimulation waveforms to the target brain region, translating the stimulation parameters into electrical signals that are suitable for the stimulation electrodes. By integrating rhythmic patterns and pulse dynamics into the delivery process, the stimulation applicator 130 may activate the intended targeted neurons while minimizing unintended activation of other neurons. For example, a stimulation protocol designed for interneurons may use super-threshold pulses at frequencies that may selectively engage this neural type, avoiding significant activation of pyramidal neurons. Similarly, for stroke rehabilitation, the stimulation applicator 130 may target the motor cortex with a super-threshold stimulation to facilitate the recovery of motor functions. Additionally, unsupervised machine-learning algorithms, such as clustering techniques, may evaluate neural response patterns during a therapy to identify optimal stimulation parameters, minimizing the risk of overstimulation or neural fatigue. Working in tandem, these components enable the super-threshold stimulator 505 to perform precise, frequency-specific stimulation that activates targeted neurons.

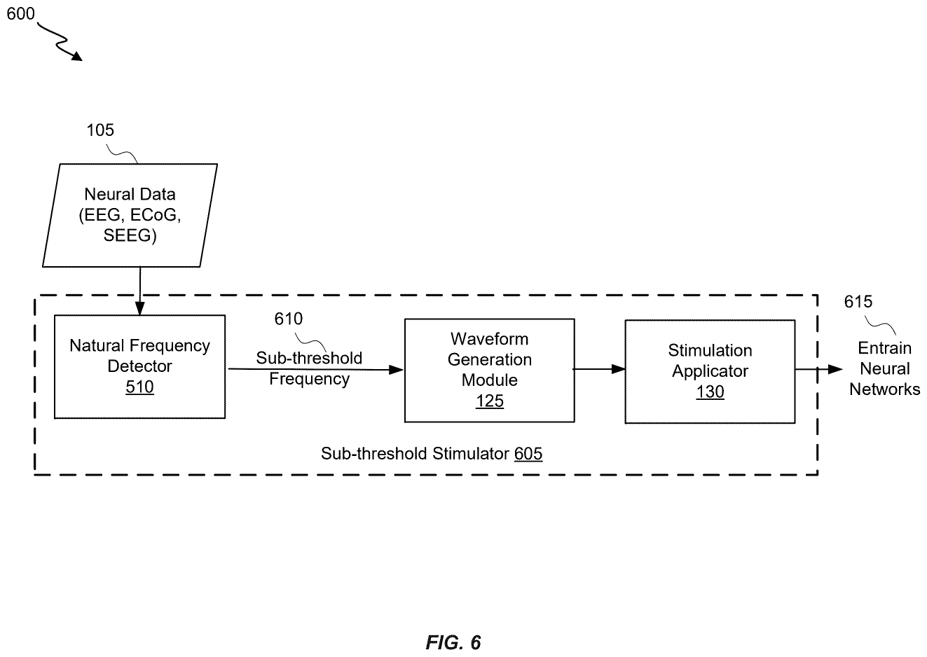

FIG. 6 shows an example implementation 600 of a sub-threshold stimulator configured to entrain neural networks in accordance with some embodiments of the present disclosure. The sub-threshold stimulator 605 may include the natural frequency detector 510, the waveform generation module 125, and the stimulation applicator 130. The natural frequency detector 510 may identify the natural frequencies of the targeted neurons by analyzing neural activity to generate a sub-threshold frequency 610. Once identified, the sub-threshold frequency 610 may be passed to the waveform generation module 125, that may create waveforms tailored to the sub-threshold frequency 610.

Sub-threshold waveform may have an amplitude below the level required to directly elicit action potentials but may still influence neural firing patterns. For example, a sub-threshold sine wave at 25 Hz may align closely with the natural frequency of interneurons, enhancing their firing likelihood during waveform peaks and reducing it during troughs. Stimulation induced, using such waveform, may increase the relative activity of interneurons while maintaining a minimal engagement of pyramidal neurons that may have different natural frequencies. For instance, in cases of anxiety disorders, sub-threshold stimulation at theta band frequencies (4-8 Hz) may help regulate hyperactivity in the amygdala, a region often associated with heightened emotional responses. Machine-learning models, such as long short-term memory (LSTM) networks, may be configured to evaluate the neural data over time to detect various patterns of hyperactivity and recommend optimal sub-threshold frequencies tailored to each subject. Additionally, reinforcement learning algorithms may be used to adjust stimulation parameters dynamically to maximize network entrainment without overstimulation.

Finally, the stimulation applicator 130 may deliver the generated sub-threshold waveform to the target region of the brain of a subject. The application of such precise, frequency-specific stimulation may enhance the entrainment of the neural network, aligning the oscillatory activity of the targeted neurons with the delivered sub-threshold waveform. As a result, the network may adopt the rhythmic activity of the sub-threshold waveform, enabling preferential engagement of certain neurons, such as interneurons, over others. For example, during rehabilitation of the subjects for a stroke recovery, sub-threshold stimulation may be used to retrain motor cortex networks by gradually entraining them to desired frequencies, which facilitate in the functional recovery. Unsupervised clustering algorithms may evaluate responses from adjacent neural network to fine-tune the stimulation, enabling targeted engagement of damaged areas while avoiding overstimulation of healthy regions. Moreover, sub-threshold stimulation may also be employed to precondition neural networks before a super-threshold stimulation, creating an optimal environment for subsequent therapeutic interventions.

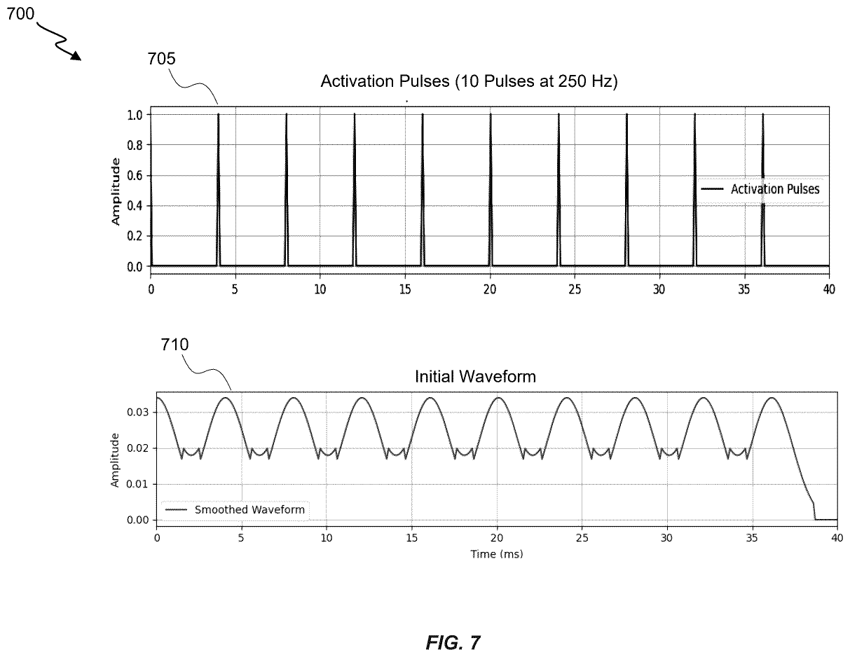

FIG. 7 shows example plots of the initial waveform and the stimulation pulses based on the baseline activity thresholds of the target neurons in accordance with some embodiments of the present disclosure. The plot 705 may show the stimulation pulses, with the X-axis representing time (in milliseconds) and the Y-axis representing amplitude or intensity of the neural signal. The plot 705 may depict 10 discrete activation pulses (also referred herein as the stimulation pulses), each occurring at a frequency of 250 Hz, meaning that the pulses may occur at regular intervals of 4 milliseconds. The stimulation pulses, appearing as vertical lines at specific time points, may represent the high-frequency neural activation, and the amplitude of each spike signifies the magnitude of the stimulation at each instance. The spacing between the pulses may reflect the estimated timing of neural activations, which is important in triggering the subsequent modulation processes.

The plot 710 may show the conversion of the stimulation pulses into the initial waveform, which is a smoother, continuous signal. The initial waveform may be formed by applying a filtering or envelope-shaping technique to the sharp activation pulses. As a result, the discrete spikes may merge into a smoother curve that represents the overall pattern of neural activation over time. The smoothing process may help visualize the high-frequency activation pulses combined to form a more structured and continuous signal, that may serve as the foundation for further modulation in subsequent layers of the signal's evolution.

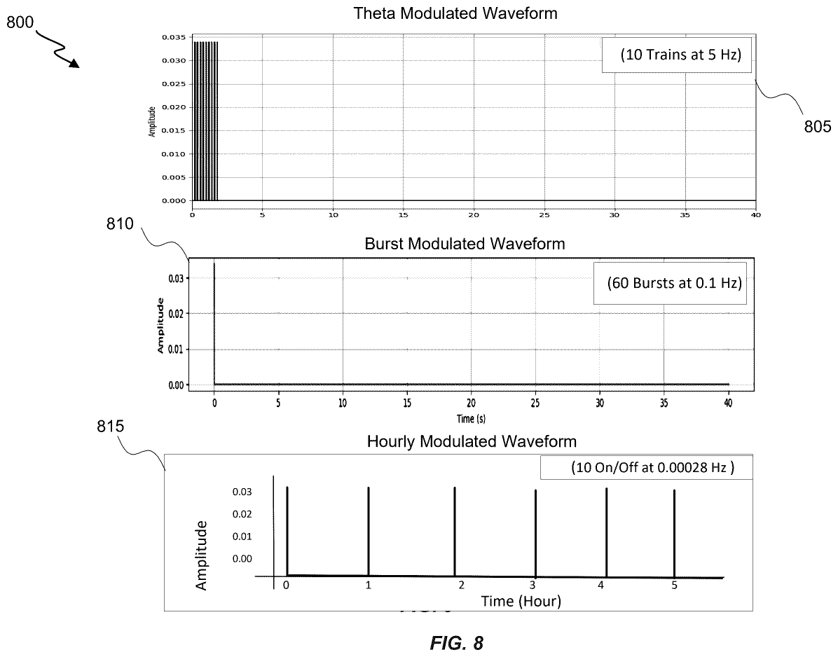

FIG. 8 shows example plots of the initial waveform modulated over time based on varying modulation schemes according to some embodiments of the present disclosure. Plot 805 may depict a graphical representation of the theta modulated waveform, comprising 10 trains at 5 Hz. The theta modulated waveform may be shown by a series of rapid bursts, with each train representing a sequence of high-frequency pulses delivered at 5 Hz (5 times per second). These bursts may be spaced periodically, forming a rhythmic pattern corresponding to the theta frequency range, typically associated with cognitive states such as relaxation and focused attention. Furthermore, the theta modulated waveform may exhibit slight variations within this frequency range to accommodate individual neural responses. The train duration and spacing between bursts may be adjusted according to the specific requirements of the therapeutic intervention.

The plot 810 may represent the burst modulated waveform, that comprises of 60 bursts at 0.1 Hz. The burst modulated waveform features bursts of stimulation delivered at a low frequency of 0.1 Hz (one burst every 10 seconds). The bursts can be arranged in a periodic sequence, with each burst potentially involving a series of high-frequency pulses, typically aimed at activating targeted neurons. The on/off cycles (also referred herein as the duty cycles) of this waveform can be tailored to allow for periods of rest between stimulation bursts, promoting neuroplasticity and minimizing the risk of overstimulation. The burst duration and rest periods may vary depending on the needs of the subject and the specific therapeutic objective. Finally, the plot 815 may illustrate an hourly modulated waveform, featuring 10 duty cycles at 0.00028 Hz. Each duty cycle in the plot 815 may represent an on/off cycle delivered once every hour over a 10-hour period. The frequency of the duty cycles can be set at an exceptionally low value of 0.00028 Hz, meaning that each cycle occurs once per hour. These low-frequency cycles may allow for sufficient rest periods between stimulation sessions to avoid overstimulation and to sustain long-term engagement of the targeted neurons. The on/off duration within each cycle can be adjusted within certain ranges, improving the therapeutic effect.

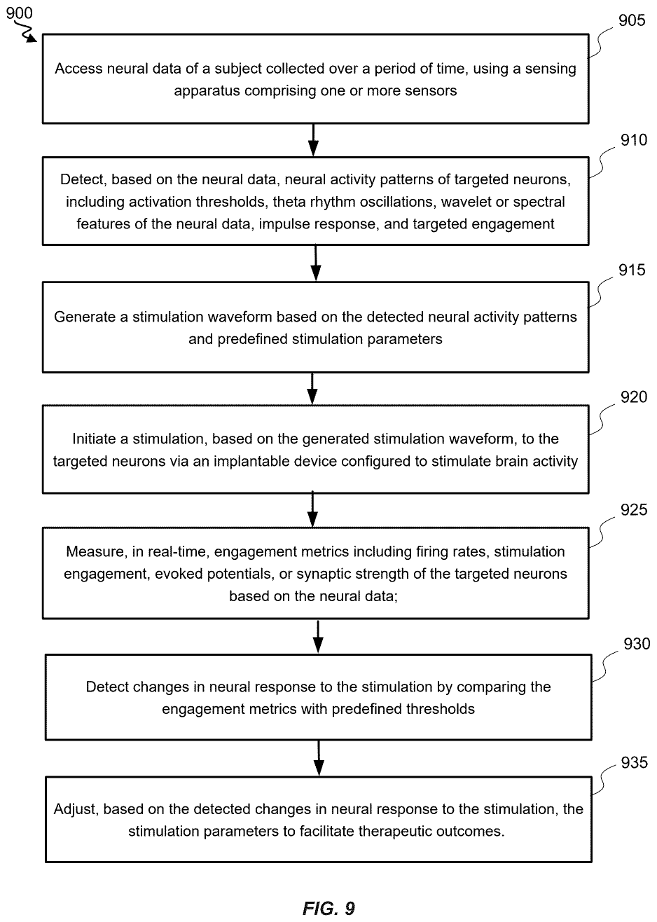

FIG. 9 shows an example flowchart for generating a stimulation waveform based on the neural data in accordance with some embodiments of the present disclosure. At block 905, the neural data of a subject may be accessed using an apparatus that may include one or more sensors. The one or more sensors may be mounted on the dural surface of the skull of the subject, such as EEG, EOG, sEEG, US, or fNIRS sensors. Following the collection of neural data 105, neural activity patterns associated with the neural data 105 may be identified at block 910. The neural activity patterns may include impulse response, theta rhythms, natural frequencies, and/or activation thresholds. Machine-learning models, such as recurrent neural networks (RNNs) or support vector machines (SVMs), may be used to analyze the data to classify neural activity patterns specific to conditions like Parkinson's disease or depression.

At block 915, a stimulation waveform may be generated comprising a plurality of stimulation pulses with distinct frequencies based on the detected neural activity patterns and predefined stimulation parameters. The stimulation parameters may include the identified neural activity patterns and schedule cycles, such as burst cycles, duty cycles, or hourly and daily durations. In some aspects, the schedule cycles may be defined by the user through a user interface or determined automatically using machine-learning models, such as reinforcement learning models that may iteratively refine the stimulation parameters based on the feedback from the prior sessions. For instance, in stroke rehabilitation, ML algorithms may adjust burst durations to maximize motor recovery. Thus, stimulation pulses generated based on the frequency components, determined through the neural activity patterns, and schedule cycles may be aligned or synchronized together to generate the stimulation waveform.

At block 920, the generated stimulation waveform may be used to initiate an externally powered cortical stimulation (XCS) to the targeted neurons via one or more stimulation electrodes having one or more configurations. The configurations of the stimulation electrodes may include a single dural-facing electrode for localized stimulation or multiple shorted electrodes for a broader activation, as in cases of generalized neurological disorders.

Subsequently, at block 925, engagement metrics including firing rates, synaptic strength, and the natural frequencies of the targeted neurons may be recorded or measured using the one or more sensors, for further accessing the neuroplastic changes induced by the stimulation. At block 930, measured activity metrics may be compared with predefined thresholds to analyze the induced neuroplastic changes such that the predefined thresholds may correspond to expected ranges of the firing rates, synaptic strength, and natural frequencies associated with successful plasticity induction. The analyses may guide a user or a clinician to adjust the stimulation parameters, including future duty cycles tailored to the subject's neural dynamics; alternatively, these adjustments may be automatically implemented by a computing system, at block 935. The adjustments may involve modifying the stimulation duration, frequency, or intensity to optimize outcomes. Additionally, the analysis may enable the generation of updated stimulation waveforms to maintain or enhance the established neuroplasticity. Once neuroplasticity is firmly established, the system may dynamically reduce or discontinue daily therapy cycles, enabling efficient and sustained therapeutic effects for conditions like Alzheimer's or chronic pain.

Some embodiments of the present disclosure include a system comprising one or more data processors. In certain embodiments, the system comprises a non-transitory computer-readable storage medium storing instructions that, when executed by one or more data processors, cause the one or more data processors to execute at least a portion of one or more methods and/or processes disclosed herein. In some embodiments, the present disclosure provides a computer program product tangibly embodied in a non-transitory machine-readable storage medium, the product comprising instructions that, when executed by one or more data processors, cause the one or more data processors to perform at least a portion of one or more methods and/or processes as disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability, or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In some instances, well-known circuits, processes, algorithms, structures, and techniques may be shown without unnecessary detail in order to avoid obscuring the embodiments.

1. A computer-implemented method including:
accessing neural data of a subject collected over a period of time, using an apparatus that included one or more sensor electrodes positions on a dural surface near targeted neurons;
identifying one or more baseline activity metrics including a baseline firing rate, a baseline stimulation engagement, or a baseline synaptic strength of the targeted neurons based on the neural data;
detecting, based on the neural data, one or more neural activity patterns of the targeted neurons, wherein the neural activity patterns include an activation threshold, a magnitude of a theta rhythm oscillations, a wavelet or spectral feature of the neural data, an impulse response, or a metric of engagement of the targeted neurons;
generating a stimulation waveform based on the one or more detected neural activity patterns and based on one or more predefined stimulation parameters, wherein the one or more predefined stimulation parameters include an amplitude, a duty cycle, a burst cycle, an hourly cycle, or a daily cycle;
initiating a stimulation, based on the generated stimulation waveform, via one or more stimulation electrodes, wherein the apparatus includes the one or more stimulation electrodes;
measuring, in real-time, other neural data during or after the stimulation using the apparatus;
identifying one or more activity metrics including a firing rate, a metric of stimulation engagements, or a synaptic strength of the targeted neurons based on the other neural data;
detecting a change in a neural response by comparing the one or more activity metrics with the one or more baseline activity metrics; and
adjusting, based on the detected changes in neural response, the stimulation parameters.

2. The computer-implemented method of claim 1, wherein the generation of the stimulation waveform includes:
generating a set of stimulation pulses based on the impulse response and the activation threshold of the targeted neurons; and
generating a waveform from the set of stimulation pulses, wherein the waveform:
includes a smoothed signal derived from the set of stimulation pulses;
modulating the waveform to include the burst cycle comprising a plurality of trains, and wherein the burst cycle is delivered at a frequency that aligns with the theta rhythm oscillations of the targeted neurons;
modulating the waveform to include the duty cycle comprising a plurality of burst cycles, each delivered 5 to 15 minutes every hour for facilitating changes in neural activity;
modulating the waveform to include the hourly cycle for facilitating dendritic spines formation in the targeted neurons.

3. The computer-implemented method of claim 1, wherein the stimulation waveform includes a component between 2-50 Hz.

4. The computer-implemented method of claim 1, further comprising:
analyzing baseline neural activity patterns from the neural data to predict a minimal stimulation level for activation of the targeted neurons to identify the activation threshold;
probing the targeted neurons with short stimulation bursts to identify the impulse response;
determining the theta rhythm by:
analyzing, from the neural data, frequency components within a range of 3 to 8 Hz;
computing, based on the frequency components, a power spectral density; and
selecting a frequency within one standard deviation of a peak power in the power spectral density to obtain the theta rhythm.

5. The computer-implemented method of claim 4, wherein the change in the response of the targeted neurons was due to neuroplasticity.

6. The computer-implemented method of claim 4, wherein the change in the response of the targeted neurons was due to a change in posture.

7. The computer-implemented method of claim 4, wherein the change in the response of the targeted neurons was due to neurodegeneration.

8. The computer-implemented method of claim 1, wherein the real-time measurement of engagement metrics further comprises:
determining the firing rate by detecting number of action potentials (spikes) within a defined time window using spike-sorting algorithms, spectral analysis, or wavelet transforms to isolate neural firing events from the neural data; and
estimating the synaptic strength by analyzing variations in amplitude and duration of the neural data collected over a period of time using time-series decomposition, short-time Fourier transforms (STFT), or convolutional neural networks (CNNs) configured to classify synaptic efficacy based on the neural activity patterns.

9. The computer-implemented method of claim 8, wherein the spectral analysis further includes:
analyzing a spectral power in gamma band (30-100 Hz) or high-gamma band (>100 Hz) from the neural data;
detecting changes in spectral power as an indicator of neural response to stimulation; and
adjusting stimulation parameters based on the detected spectral power changes.

10. The computer-implemented method of claim 1, wherein the detection of neuroplastic changes induced by the stimulation is based on an increase in the firing rate of the targeted neurons by at least 20%, an increase in synaptic strength by at least 10%, or synchronization of theta rhythm oscillations with a frequency of the stimulation waveform by at least 70%.

11. The computer-implemented method of claim 1, wherein the one or more sensors include an electroencephalography (EEG) electrode, functional near infrared spectroscopy (fNIRS) sensor, ultrasound (US) sensor, electrocorticography (ECoG) electrode, epidural electrocorticography electrodes electrode, or stereotactic electroencephalography (sEEG) electrode.

12-23. (canceled)

24. A computer-program product tangibly embodied in a non-transitory machine-readable storage medium, including instructions configured to cause one or more data processors to perform:
accessing neural data of a subject collected over a period of time, using an apparatus that included one or more sensor electrodes positions on a dural surface near targeted neurons;
identifying one or more baseline activity metrics including a baseline firing rate, a baseline stimulation engagement, or a baseline synaptic strength of the targeted neurons based on the neural data;
detecting, based on the neural data, one or more neural activity patterns of the targeted neurons, wherein the neural activity patterns include an activation threshold, a magnitude of a theta rhythm oscillations, a wavelet or spectral feature of the neural data, an impulse response, or a metric of engagement of the targeted neurons;
generating a stimulation waveform based on the one or more detected neural activity patterns and based on one or more predefined stimulation parameters, wherein the one or more predefined stimulation parameters include an amplitude, a duty cycle, a burst cycle, an hourly cycle, or a daily cycle;
initiating a stimulation, based on the generated stimulation waveform, via one or more stimulation electrodes, wherein the apparatus includes the one or more stimulation electrodes;
measuring, in real-time, other neural data during or after the stimulation using the apparatus;
identifying one or more activity metrics including a firing rate, a metric of stimulation engagements, or a synaptic strength of the targeted neurons based on the other neural data;
detecting a change in a neural response by comparing the one or more activity metrics with the one or more baseline activity metrics; and
adjusting, based on the detected changes in neural response, the stimulation parameters.

25. The computer-program product of claim 24, wherein the generation of the stimulation waveform includes:
generating a set of stimulation pulses based on the impulse response and the activation threshold of the targeted neurons; and
generating a waveform from the set of stimulation pulses, wherein the waveform:
includes a smoothed signal derived from the set of stimulation pulses;
modulating the waveform to include the burst cycle comprising a plurality of trains, and wherein the burst cycle is delivered at a frequency that aligns with the theta rhythm oscillations of the targeted neurons;
modulating the waveform to include the duty cycle comprising a plurality of burst cycles, each delivered 5 to 15 minutes every hour for facilitating changes in neural activity;
modulating the waveform to include the hourly cycle for facilitating dendritic spines formation in the targeted neurons.

26. The computer-program product of claim 24, wherein the stimulation waveform includes a component between 2-50 Hz.

27. The computer-program product of claim 24, wherein the set of actions further include:
analyzing baseline neural activity patterns from the neural data to predict a minimal stimulation level for activation of the targeted neurons to identify the activation threshold;
probing the targeted neurons with short stimulation bursts to identify the impulse response;
determining the theta rhythm by:
analyzing, from the neural data, frequency components within a range of 3 to 8 Hz;
computing, based on the frequency components, a power spectral density; and
selecting a frequency within one standard deviation of a peak power in the power spectral density to obtain the theta rhythm.

28. The computer-program product of claim 27, wherein the change in the response of the targeted neurons was due to neuroplasticity.

29. The computer-program product of claim 27, wherein the change in the response of the targeted neurons was due to a change in posture.

30. The computer-program product of claim 27, wherein the change in the response of the targeted neurons was due to neurodegeneration.

31. The computer-program product of claim 24, wherein the real-time measurement of engagement metrics further comprises:
determining the firing rate by detecting number of action potentials (spikes) within a defined time window using spike-sorting algorithms, spectral analysis, or wavelet transforms to isolate neural firing events from the neural data; and
estimating the synaptic strength by analyzing variations in amplitude and duration of the neural data collected over a period of time using time-series decomposition, short-time Fourier transforms (STFT), or convolutional neural networks (CNNs) configured to classify synaptic efficacy based on the neural activity patterns.

32. The computer-program product of claim 31, wherein the spectral analysis further includes:
analyzing a spectral power in gamma band (30-100 Hz) or high-gamma band (>100 Hz) from the neural data;
detecting changes in spectral power as an indicator of neural response to stimulation; and
adjusting stimulation parameters based on the detected spectral power changes.

33. The computer-program product of claim 24, wherein the detection of neuroplastic changes induced by the stimulation is based on an increase in the firing rate of the targeted neurons by at least 20%, an increase in synaptic strength by at least 10%, or synchronization of theta rhythm oscillations with a frequency of the stimulation waveform by at least 70%.

34. The computer-program product of claim 24, wherein the one or more sensors include an electroencephalography (EEG) electrode, functional near infrared spectroscopy (fNIRS) sensor, ultrasound (US) sensor, electrocorticography (ECoG) electrode, epidural electrocorticography electrodes electrode, or stereotactic electroencephalography (sEEG) electrode.

35. A system comprising:
one or more processors;
one or more non-transitory computer-readable media storing instructions, which, when executed by the system, cause the system to perform a set of actions including:
accessing neural data of a subject collected over a period of time, using an apparatus that included one or more sensor electrodes positions on a dural surface near targeted neurons;
identifying one or more baseline activity metrics including a baseline firing rate, a baseline stimulation engagement, or a baseline synaptic strength of the targeted neurons based on the neural data;
detecting, based on the neural data, one or more neural activity patterns of the targeted neurons, wherein the neural activity patterns include an activation threshold, a magnitude of a theta rhythm oscillations, a wavelet or spectral feature of the neural data, an impulse response, or a metric of engagement of the targeted neurons;
generating a stimulation waveform based on the one or more detected neural activity patterns and based on one or more predefined stimulation parameters, wherein the one or more predefined stimulation parameters include an amplitude, a duty cycle, a burst cycle, an hourly cycle, or a daily cycle;
initiating a stimulation, based on the generated stimulation waveform, via one or more stimulation electrodes, wherein the apparatus includes the one or more stimulation electrodes;
measuring, in real-time, other neural data during or after the stimulation using the apparatus;
identifying one or more activity metrics including a firing rate, a metric of stimulation engagements, or a synaptic strength of the targeted neurons based on the other neural data;
detecting a change in a neural response by comparing the one or more activity metrics with the one or more baseline activity metrics; and
adjusting, based on the detected changes in neural response, the stimulation parameters.
